(19) United States
(12) Patent Application Publication
(10) Pub. No.: US 2021/0009684 A1
(43) Pub. Date: Jan. 14, 2021

(54) THERAPEUTIC TARGETING OF TISSUE-RESIDENT MEMORY T CELLS

(71) Applicant: REGENTS OF THE UNIVERSITY OF MINNESOTA, Minneapolis, MN (US)

(72) Inventors: David Masopust, New Brighton, MN (US); Vaiva Vezys, New Brighton, MN (US)

(21) Appl. No.: 16/922,734

(22) Filed: Jul. 7, 2020

Related U.S. Application Data
(60) Provisional application No. 62/871,442, filed on Jul. 8, 2019.

(51) Int. Cl. C07K 16/28

(52) U.S. Cl. CPC C07K 16/28; A61K 45/06

(57) ABSTRACT

Provided are methods that include administering a composition to a subject suffering from a disease or condition, wherein the composition includes a compound that binds to a resident memory T cell (TRM) marker. The method may include depleting the numbers of TRM in the area where the composition was delivered. Such a depletion may result in an improvement in a disease or condition including an inflammatory immune response that is regulated or sustained by the TRM. Also provided are compositions including a compound that binds to a TRM marker. Such compositions may be used in the treatment of a disease or condition including an inflammatory immune response.

### CONTINUING APPLICATION DATA

This application claims the benefit of U.S. Provisional Application Ser. No. 62/871,442, filed Jul. 8, 2019 which is incorporated by reference herein.

### BACKGROUND

Resident memory T cells (also referred to as tissue-resident memory T cells or TRM) are a lymphocyte lineage that constitutes the most abundant antigen-experienced T cell population in humans. TRM are absent in blood; rather, they are located in tissues through the rest of the body, making them difficult to isolate and characterize. Because of these difficulties, TRM were not discovered until recently—long after the discovery and characterization of many other T cell populations.

### SUMMARY OF THE INVENTION

In one aspect, this disclosure describes a method that includes administering a composition to a subject suffering from a disease or condition, wherein the composition includes a compound that binds to a resident memory T cell (TRM) marker. In some embodiments, the method preferably includes depleting the numbers of TRM in the area where the composition was delivered. In some embodiments, such a depletion may result in an improvement in a disease or condition including an inflammatory immune response that is regulated or sustained by the TRM.

In another aspect, this disclosure describes a composition that includes a compound that binds to a TRM marker, wherein the composition is formulated for oral, rectal, colonic, vaginal, topical, nasal, ophthalmic, and/or parenteral administration. In some embodiments, the compound that binds to a TRM marker includes an antibody. In some embodiments, the antibody fixes complement, induces antibody dependent cell cytotoxicity (ADCC), and/or induces phagocytosis of a TRM.

In a further aspect, this disclosure describes a composition including a compound that binds to a TRM marker for use in the treatment of a disease or condition including an inflammatory immune response.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The terms “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

Unless otherwise specified, “a,” “an,” “the,” and “at least one” are used interchangeably and mean one or more than one.

As used herein, the term “or” is generally employed in its usual sense including “and/or” unless the content clearly dictates otherwise.

The term “and/or” means one or all of the listed elements or a combination of any two or more of the listed elements.

Also herein, the recitations of numerical ranges by endpoints include all numbers subsumed within that range (for example, 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

All headings are for the convenience of the reader and should not be used to limit the meaning of the text that follows the heading, unless so specified.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

### BRIEF DESCRIPTION OF THE DRAWINGS

The patent or application file contains drawings and photographs executed in color. Copies of this patent or patent application publication with color drawings and photographs will be provided by the Office upon request and payment of the necessary fee.

FIG. 1A-FIG. 1C show CD69 and CD103 expression are not sufficient to infer residence. FIG. 1A. Lymphocytic choriomeningitis virus (LCMV)-specific (P14) CD8⁺ T cells (5×10⁵) were transferred to B6 mice, and 1 day later were infected with LCMVArmstrong. At >30 days after infection (memory), mice were infected with vesicular stomatitis virus (Indiana strain) (VSV) or were not infected. Two days later mice were sacrificed, and P14 cells in nonlymphoid (NLT) and secondary lymphoid (SLO) tissues were analyzed for CD69, CD103, and CD62L expression by fluorescence flow cytometry. Shown are representative images from the female reproductive tract (FRT) and spleen (SPL). Percentages are based on total P14 cells. FIG. 1B. Experimental scheme for testing residence of CD8⁺ T cells in B6 mice that have been co-housed with pet store mice. FIG. 1C. Representative images of the phenotype of host and partner CD44^{hi} CD8⁺ T cells in pooled inguinal and mesenteric LNs (lymph nodes). FIG. 1C adapted from Beura et al. (Beura et al. Immunity 48, 327-338 e325 (2018)).

FIG. 2A-FIG. 2B show an exemplary workflow for the identification of putative core TRM biomarkers and tissue-specific CD8⁺ TRM biomarkers by RNA sequencing and differential expression analysis of several TRM populations, circulating memory T cells, and cells that share some markers with TRM even though they are not TRM (for example, cells that express CD69 by recent activation or by bystander activation). FIG. 2A. LCMV specific (P14) cells (5×10⁵) were transferred to B6 mice, and 1 day later the mice were infected with LCMV-Armstrong. These are called P14 chimeras. At day 4.5 after infection, some mice were sacrificed and effector P14 cells were isolated by fluorescence-activated cell sorting (FACS) of splenocytes. At >30 days after infection, mice were infected with VSV or were not infected. Two days later, bystander activated P14 cells were isolated by FACS of splenocytes from mice infected with VSV. Mice not infected with VSV were sacrificed and FACS was used to isolate female reproductive tract (FRT), small intestine intraepithelial lymphocytes (IEL), small intestine lamina propria (LP), liver (LIV), and spleen (SPL) resident P14 cells. FIG. 2B. Resident, circulating, and masquerading CD8⁺ T cell subsets were sequenced, and pair-wise comparisons were made to identify core (common) and tissue-specific resident gene signatures.

FIG. 3A-FIG. 3B show exemplary approaches for validating putative TRM biomarkers. FIG. 3A. As described in the description of FIG. 1, P14 memory chimeras were created and infected with VSV (to induce bystander activation of P14 memory cells) or were not infected. Two days later mice were sacrificed and P14 cells in the FRT and SPL were analyzed by fluorescence flow cytometry for CD69, and a putative TRM biomarker (for example, P2rx7), and CD62L expression. FIG. 3B. Additionally or alternatively, the applicability of a putative TRM biomarker (for example, P2rx7) may be evaluated by parabiosis studies in dirty mice.

FIG. 4A-FIG. 4D show local depletion of epidermal TRM cells by treatment with an antibody. FIG. 4A. A virus model was used to establish virus (VSV-OVA)-specific CD8⁺ resident memory cells (OT-I) in the epidermis of mice. The OT-I cells expressed Thy1.1. To test for depletion, anti-Thy1.1 (HIS51) antibody was intradermally (i.d.) injected into one flank while the same volume of isotype-control antibody was injected into the opposite flank. FIG. 4B. Enumeration of OT-I cells in the epidermis 7 days after i.d. injections. FIG. 4C. Representative images of the epidermis. FIG. 4D. Representative images of spleen (SPL), small intestine (SI), and salivary gland (SG).

FIG. 5A-FIG. 5C shows the TRM depletion that results from local injection of an antibody targeting CD103 into the skin of a mouse. Subsets of TRM expression CD103. FIG. 5A. Skin was stained with a fluorescent antibody (teal) that targets CD45.1, which, in these mice, is only expressed on OTI T cells. Quantified results are shown in FIG. 5B. FIG. 5C shows representative images of immunofluorescence staining (top) and counts (bottom) of skin 21 days post-local treatment with anti-Thy1.1 or anti-CD103 antibody.

FIG. 6 shows the TRM depletion that results from local injection of an antibody targeting CD49a into the skin of a mouse. Skin was stained with a fluorescent antibody (teal) that targets CD45.1, which, in these mice, is only expressed on OTI T cells. CD8β staining is in red. Staining for nuclei is in dark blue.

FIG. 7A shows expression values as counts per million (CPM) for P2rx7. FRT, Female Reproductive Tract; IEL, Small Intestine Intraepithelial lymphocytes; LP, Small Intestine lamina propria lymphocytes; SP, Spleen; CIR, Circulating Memory Subsets; TEM, Effector Memory T cells; TCM, Central Memory T cells; MASQ, masquerading subset (CD69⁺ non-TRM); BYS, Bystanders; LCMVD4, four days after lymphocytic choriomeningitis virus (LCMV) infection. FIG. 7B shows validation of P2rx7 expression using flow cytometry on lymphocytic choriomeningitis virus (LCMV)-specific cells (P14s).

FIG. 8 shows expression of TRM markers on non-TRM CD8⁺ T cells in the context of VSV-induced bystander activation. P14 cells from the SPL (spleen), FRT (female reproductive tract), and SI-IEL (small intestine intraepithelial lymphocytes) were analyzed for TRM marker expression by fluorescence flow cytometry as further described in Example 4. Cells were gated on CD62L^{hi}CD44^{lo} (Naïve), LCMV-specific memory gp33 tetramer⁺ CD62L^{hi}CD44⁺ (TCM), or gp33 tetramer⁺ cells (IEL, FRT).

FIG. 9 shows TRM markers are induced by chronic infection. Fluorescence flow cytometry plots of gp33 tetramer⁺ CD8⁺ T cells of the blood, SPL (spleen), FRT (female reproductive tract), SI-IEL (small intestine intraepithelial) and SI-LP (small intestine lamina propria) were analyzed as further described in Example 5.

### DETAILED DESCRIPTION OF ILLUSTRATIVE EMBODIMENTS

This disclosure describes compositions including a compound that binds to a resident memory T cell (TRM) biomarker and methods of using those compositions. For example, the compositions may be administered, as further described herein to locally deplete TRM in a tissue.

Tissue-resident memory CD4⁺ and CD8⁺ T cells have been identified in many tissues and organs of mice and humans (Carbone J Immunol 195, 17-22 (2015), Schenkel et al. J Immunol 192, 2961-2964 (2014), Schenkel et al. Immunity 41, 886-897 (2014), Jameson et al. Immunity 48, 214-226 (2018)). While TRM that reside in barrier (for example, skin, gut, lung, etc.) and non-barrier tissues (for example, pancreas, kidney, liver, brain, etc.) play an essential role in protecting against infections and cancer, aberrant activation of these cells may give rise to allergic and autoimmune (AI) conditions, including asthma, inflammatory bowel disease, vitiligo, and multiple sclerosis (Clark Sci Transl Med 7, 269rv261 (2015), Wu et al. Autoimmun Rev 17, 906-911 (2018)). Depletion of local TRM are a potential therapeutic strategy for allergies and autoimmune diseases that are T cell driven. However, one major impediment in developing strategies for depleting TRM is a dearth of reliable TRM markers. To date, there are only a few TRM associated markers that are identifiable by flow cytometry and rigorously associated with residence (Jameson et al. Immunity 48, 214-226 (2018)). CD69, one commonly used marker of TRM, is also a marker of T cell responses to antigen or inflammation. Indeed, it has been shown that CD69 expression does not suffice to assume residence (Beura et al. Immunity 48, 327-338 e325 (2018)) and interpretations from human studies that rely on CD69 as a marker of TRM are not unequivocal. CD103, another commonly used marker of TRM is not expressed by many TRM (Topham et al. Front Immunol 9, 515 (2018)).

In one aspect, this disclosure describes a composition including a compound that binds to a TRM biomarker (that is, a molecule expressed on the surface of a TRM). In another aspect, this disclosure describes a method that includes administering the composition including a compound that binds to a TRM biomarker to a subject.

In some embodiments, the TRM biomarker is not expressed on a non-TRM (for example, a bystander activated) T cell. As shown in Example 4, CD101 is an exemplary TRM biomarker that is not expressed non-TRM T cells.

In some embodiments, the TRM biomarker includes CD103, CD49a, CCR8, CD38, CD39, NKG2D, CD69, CD101, PD-1, P2rx7, IL-23R, CD4, or CD8. In some embodiments, the TRM biomarker includes a combination of TRM biomarkers. For example, the TRM biomarker may include two, three, four, or more of CD103, CD49a, CCR8, CD38, CD39, NKG2D, CD69, CD101, PD-1, P2rx7, IL-23R, CD4, or CD8. For example, in some exemplary embodiments, the TRM biomarker could include CD103 and NKG2D; CD38 and P2rx7; IL-23R and CD4; CD49a, NKG2D, and CD101; or CD103, CD49a, and CD8.

As shown in Example 1, biomarkers for TRM may be tissue specific. For example, although a few non-TRM cells express CD38, CD38 may be used as an exemplary tissue-specific TRM biomarker. Additionally, as shown in Example 5, biomarkers for TRM may be disease or condition specific. When, as further described below, the method includes treating a disease or condition, the compound that binds to a TRM biomarker may be selected based on specific TRM biomarkers present in that disease or condition.

In some embodiments, the compound that binds to a TRM biomarker includes an antibody to the TRM biomarker. An antibody to a TRM biomarker may include an antibody that fixes complement, an antibody that induces antibody dependent cell cytotoxicity (ADCC), and/or an antibody that induces phagocytosis of the TRM. In some embodiments, an antibody to the TRM biomarker preferably causes depletion of the TRM including, for example, by fixing complement, inducing ADCC, and/or inducing phagocytosis of the TRM.

In some embodiments, the compound that binds to a TRM biomarker includes a diabody that includes a portion of the compound that facilitates depletion of the TRM in addition to a portion of the compound that binds to the TRM biomarker. For example, a compound that facilitates depletion of the TRM may fix complement, induce ADCC, induce phagocytosis of the TRM, and/or bind to a CD8⁺ T cell or NK cell.

In some embodiments, the method includes administering the composition to a subject suffering from a disease or condition. In some embodiments, a disease or condition may include an inflammatory immune response including a dysregulated inflammatory immune response. For example, the disease or condition may include an autoimmune disease or an allergic disease. Exemplary diseases or conditions include alopecia areata, asthma, arthritis (including rheumatoid arthritis), multiple sclerosis, psoriasis, Type I diabetes, inflammatory bowel disease (IBD), graft versus host disease (GVHD), Sjogren's disease, and rejection following solid organ transplantation.

The subject may include a vertebrate, more preferably a mammal, such as a human patient, or an animal. An animal may include a companion animal, a research animal, a domesticated animal, or an animal in the wild. Companion animals include, but are not limited to, dogs, cats, hamsters, gerbils, and guinea pigs. Domesticated animals include, but are not limited to, cattle, horses, pigs, goats, and llamas. Research animals include, but are not limited to, mice, rats, dogs, apes, and monkeys.

In some embodiments, the method may include evaluating the subject for an improvement in the disease or condition. Improvement may include a decrease the severity of the symptoms of one of the disease or condition, or completely removing the symptoms of the disease or condition.

Without wishing to be bound by theory, a treatment that depletes TRM is expected to remove a resident instigator of chronic or relapsing local inflammatory diseases (that is, the TRM), resulting in remission. The recent discovery of a role for TRM as local “master regulators” of inflammatory immune responses suggests that disrupting TRM's capacity to regulate or sustain pathology in many autoimmune and allergic diseases may have a therapeutic effect.

As shown in Examples 1 and 2, biologics that target TRM biomarkers can deplete local TRM in vivo. Proof of principle studies in mouse skin successfully validated this approach for TRM depletion.

In some embodiments, the method includes locally delivering the composition. The location may be selected based on the disease or condition to be treated. For example, when the disease or condition includes alopecia areata or psoriasis, the compound that binds to the TRM biomarker may be delivered by injection into the dermis. In another example, when the disease or condition includes arthritis, the compound that binds to the TRM biomarker may be delivered by injection into a joint (that is, intraarticularly). In yet another example, when the disease or condition includes IBD, the compound that binds to the TRM biomarker may be delivered (for example, orally or rectally) in a formulation coated to dissolve at a certain pH or a certain location. In a further example, when the disease or condition includes IBD, the compound that binds to the TRM biomarker may be delivered by injection into the wall of the colon and/or rectum.

In some embodiments, the method may include locally depleting TRM. For example, when the method includes locally delivering the composition, the numbers of resident memory T cells (TRM) may be depleted in the area where the composition was delivered. In some embodiments, the method may include evaluating the subject for local depletion of TRM. When the method includes locally depleting TRM, depletion of the TRM may be measured by biopsy and histological examination or by isolating and characterizing lymphocytes by flow cytometry or enzyme-linked immune absorbent spot (ELISPOT) assay. In some embodiments when the method includes biopsy, TRM numbers may be compared between a biopsy of the treatment site and a biopsy of a distant location (for example, a biopsy of another tissue). In some embodiments, blood sampling may be used to assess TRM depletion.

In some embodiments, local depletion of TRM includes a decrease of at least 10%, at least 20%, at least 30%, at least 40%, at least 50%, at least 60%, at least 70%, at least 80%, or at least 90% of the TRM compared to the number of TRM prior to treatment with the composition.

For example, in an exemplary embodiment, when depletion of the TRM is measured by biopsy and histological examination, a decrease of at least 50% of the TRM is observed in a same biopsy area after administration of the composition compared to the numbers of TRM observed in the same biopsy area prior to administration of the composition.

In another exemplary embodiment, when depletion of the TRM is measured by biopsy and histological examination, a decrease of at least 50% of the TRM is observed in a biopsy area after administration of the composition compared to the numbers of TRM observed in an equivalent biopsy area located in a distant location (for example, in another tissue).

In yet another exemplary embodiment, when depletion of the TRM is measured by biopsy and histological examination, a decrease of at least 50% of the TRM is observed in a region defined by the limits of diffusion of the composition after administration of the composition compared to the numbers of TRM observed in an equivalently sized region located in a distant location (for example, in another tissue) and/or in the same region prior to administration of the composition.

In some embodiments, when the method includes locally depleting TRM, the method may preferably not result in a systemic change in TRM. For example, while TRM may decrease locally (for example, in a biopsy area) by at least 10%, at least 20%, at least 30%, at least 40%, at least 50%, at least 60%, at least 70%, at least 80%, or at least 90%, systemic TRM (for example, TRM outside the biopsy area) may decrease by less than 20%, more preferably less than 10%, or most preferably less than 5%.

In some embodiments, the composition may preferably be formulated for local administration. In some embodiments, the composition may be formulated for oral, rectal, colonic, vaginal, topical, nasal, ophthalmic, and/or parenteral (including, for example, subcutaneous, intramuscular, intraperitoneal, or intraarticular) administration.

In some embodiments, the composition may further include a pharmaceutically acceptable carrier. The pharmaceutically acceptable carrier may include, for example, an excipient, a diluent, a solvent, an accessory ingredient, a stabilizer, a protein carrier, or a biological compound. Non-limiting examples of a protein carrier includes keyhole limpet hemocyanin (KLH), bovine serum albumin (BSA), ovalbumin, or the like. Non-limiting examples of a biological compound which may serve as a carrier include a glycosaminoglycan, a proteoglycan, and albumin. The carrier may be a synthetic compound, such as dimethyl sulfoxide or a synthetic polymer, such as a polyalkyleneglycol. Ovalbumin, human serum albumin, other proteins, polyethylene glycol, or the like may be employed as the carrier. In some embodiments, the pharmaceutically acceptable carrier includes at least one compound that is not naturally occurring or a product of nature. In some embodiments, a composition including a the pharmaceutically acceptable carrier results in a formulation that is not naturally occurring or a product of nature.

In some embodiments, the compound that binds to a TRM biomarker may be formulated in combination with one or more additional active agents. Any known therapeutic agent may be included as an additional active agent. An exemplary additional active agent may include complement or an antibody including, for example, an antibody linked to a toxin. The action of the additional active agent in the combination therapy may be cumulative to the compound that binds to a TRM biomarker or it may be complementary, for example to manage side effects or other aspects of the subject's medical condition.

The composition may be conveniently presented in unit dosage form and may be prepared by any of the methods well-known in the art of pharmacy. Unit dosages may include or be administered from prefilled bags or syringes. In some embodiments, a method includes the step of bringing the compound that binds to a TRM biomarker into association with a pharmaceutical carrier.

A formulation suitable for oral administration may be presented as a discrete unit such as a tablet, a troche, a capsule, a lozenge, a wafer, or a cachet, containing a predetermined amount of the compound that binds to a TRM biomarker. The compound that binds to a TRM biomarker may be included as a powder or as granules, as liposomes, or as a solution or suspension in an aqueous liquor or non-aqueous liquid such as a syrup, an elixir, an emulsion, or a draught. The tablets, troches, pills, capsules, and the like can also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it can further contain a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, sugar, and the like. A syrup or elixir can contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The compound that binds to a TRM biomarker can be incorporated into preparations and devices in formulations that may be designed for sustained release. Additionally or alternatively, the formulation may be formulated for release at a particular location or under particular conditions (including, for example, at a particular pH). In some embodiments, the compound that binds to a TRM biomarker formulated for oral administration may be formulated for release in the gastrointestinal tract as described in, for example, U.S. Pat. Nos. 6,506,407 or 7,737,133.

A composition suitable for parenteral administration may include a sterile aqueous preparation including the compound that binds to a TRM biomarker, or dispersions of sterile powders including the compound that binds to a TRM biomarker, which may be preferably isotonic with the blood of the recipient. Isotonic agents that may be included in the liquid preparation include sugars, buffers, and sodium chloride. Solutions including the compound that binds to a TRM biomarker may be prepared in water, optionally mixed with a nontoxic surfactant. Dispersions including the compound that binds to a TRM biomarker may be prepared in water, ethanol, a polyol (such as glycerol, propylene glycol, liquid polyethylene glycols, and the like), vegetable oils, glycerol esters, and mixtures thereof. The dosage may preferably be sterile, fluid, and stable under the conditions of manufacture and storage. The necessary fluidity may be achieved, for example, by using liposomes, by employing the appropriate particle size in the case of dispersions, or by using surfactants. Sterilization of a liquid preparation may be achieved by any convenient method that preserves the bioactivity of the compound that binds to a TRM biomarker, preferably by filter sterilization. Preferred methods for preparing powders include vacuum drying and freeze drying of the sterile injectable solutions. Subsequent microbial contamination may be prevented using various antimicrobial agents, for example, antibacterial, antiviral and antifungal agents including parabens, chlorobutanol, phenol, sorbic acid, thimerosal, and the like.

Nasal spray formulations include purified aqueous solutions including the compound that binds to a TRM biomarker with isotonic agents. Such formulations may also include a preservative agent. Such formulations may be adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Formulations for colonic administration may include a suitable carrier such as waster, an alcohol, or an aqueous-alcohol fluid. A carrier may be thickened with natural or synthetic thickness such as gums, acrylates or modified celluloses. A formulation may also include an effective amount of a lubricant such as a natural or synthetic fat or oil, a tris-fatty acid glycerate or lecithin. Nontoxic nonionic surfactants may also be included as wetting agents and dispersants. Ophthalmic formulations may be prepared by a similar method to the nasal spray, with that the pH and isotonic factors adjusted to match that of the eye. Topical formulations may include the compound that binds to a TRM biomarker dissolved or suspended in one or more media such as mineral oil, petroleum, polyhydroxy alcohols, or other bases used for topical pharmaceutical formulations. Topical formulations may be provided in the form of a bandage, wherein the formulation is incorporated into a gauze or other structure and brought into contact with the skin. Each of these formulations may be designed for sustained release. Additionally or alternatively, the formulation may be formulated for release at a particular location or under particular conditions (including, for example, at a particular pH).

### Exemplary Method Aspects

1. A method comprising administering a composition to a subject suffering from a disease or condition, wherein the composition comprises a compound that binds to a resident memory T cell (TRM) marker.
2. The method of Aspect 1, wherein the TRM marker comprises CD103, CD49a, CCR8, CD38, CD39, NKG2D, CD69, CD101, PD-1, P2rx7, IL-23R, CD4, or CD8, or a combination thereof.
3. The method of Aspect 1 or Aspect 2, wherein the TRM marker is tissue specific.
4. The method of any one of the preceding Aspects, wherein the compound that binds to a TRM marker comprises an antibody.
5. The method of Aspect 4, wherein the antibody comprises an antibody that fixes complement, an antibody that induces antibody dependent cell cytotoxicity (ADCC), and/or an antibody that induces phagocytosis of a resident memory T cell (TRM).
6. The method any one of the preceding Aspects, wherein the disease or condition comprises an inflammatory immune response.
7. The method any one of the preceding Aspects, wherein the disease or condition comprises an autoimmune disease or an allergic disease.
8. The method any one of the preceding Aspects, wherein the disease or condition comprises alopecia areata, asthma, arthritis, multiple sclerosis, psoriasis, Type I diabetes, inflammatory bowel disease (IBD), graft versus host disease (GVHD), Sjogren's disease, or rejection following solid organ transplantation.
9. The method any one of the preceding Aspects, wherein the method further comprises evaluating the subject for improvement in the disease or condition.
10. The method any one of the preceding Aspects, wherein the method comprises local delivery of the composition.
11. The method of Aspect 10, wherein the method comprises depleting the numbers of resident memory T cells (TRM) in the area where the composition was delivered.
12. The method of Aspect 11, wherein systemic TRM are decreased by less than 20%, less than 10%, or less than 5%.
13. The method of any one of the preceding Aspects, wherein the composition is formulated for oral, rectal, colonic, vaginal, topical, nasal, ophthalmic, and/or parenteral administration.
14. The method of any one of the preceding Aspects, wherein the composition comprises a pharmaceutically acceptable carrier.
15. The method of any one of the preceding Aspects, wherein the composition further comprises an additional active agent.

### Exemplary Composition Aspects

1. A composition comprising a compound that binds to a resident memory T cell (TRM) marker, wherein the composition is formulated for oral, rectal, colonic, vaginal, topical, nasal, ophthalmic, and/or parenteral administration.
2. The composition of Aspect 1, wherein the composition comprises a pharmaceutically acceptable carrier.
3. The composition of Aspect 1 or 2, wherein the composition further comprises an additional active agent.
4. The composition of any one of the preceding Aspects, wherein the TRM marker comprises CD103, CD49a, CCR8, CD38, CD39, NKG2D, CD69, CD101, PD-1, P2rx7, IL-23R, CD4, or CD8, or a combination thereof.
5. The composition of one of the preceding Aspects, wherein the compound that binds to a TRM marker comprises an antibody.
6. The composition of Aspect 5, wherein the antibody comprises an antibody that fixes complement, an antibody that induces antibody dependent cell cytotoxicity (ADCC), and/or an antibody that induces phagocytosis of a resident memory T cell (TRM).
7. A composition comprising a compound that binds to a resident memory T cell (TRM) marker for use in the treatment of a disease or condition comprising an inflammatory immune response.
8. The composition of Aspect 7, wherein the composition is formulated for oral, rectal, colonic, vaginal, topical, nasal, ophthalmic, and/or parenteral administration.
9. The composition of Aspect 7 or Aspect 8, wherein the composition comprises a pharmaceutically acceptable carrier.
10. The composition of any one of Aspects 7 to 9, wherein the composition further comprises an additional active agent.
11. The composition of any one of Aspects 7 to 10, wherein the TRM marker comprises CD103, CD49a, CCR8, CD38, CD39, NKG2D, CD69, CD101, PD-1, P2rx7, IL-23R, CD4, or CD8, or a combination thereof.
12. The composition of any one of Aspects 7 to 11, wherein the compound that binds to a TRM marker comprises an antibody.
13. The composition of Aspect 12, wherein the antibody comprises an antibody that fixes complement, an antibody that induces antibody dependent cell cytotoxicity (ADCC), and/or an antibody that induces phagocytosis of a resident memory T cell (TRM).
14. The composition of any one of Aspects 7 to 13, wherein the disease or condition comprises an autoimmune disease or an allergic disease.
15. The composition of any one of Aspects 7 to 14, wherein the disease or condition comprises alopecia areata, asthma, arthritis, multiple sclerosis, psoriasis, Type I diabetes, inflammatory bowel disease (IBD), graft versus host disease (GVHD), Sjogren's disease, or rejection following solid organ transplantation.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Materials and Methods

Mice and infections. Memory OT-Is were generated by transferring 5×10⁴ naïve transgenic Thy1.1 + OT-I T cells into naïve C57B1/6J mice that were then infected with 2×10⁶ PFU VSV-OVA intravenously. For local epicutaneous re-challenge experiments, 0.5 μg of SIINFEKL in a volume of 30 μL was tattooed onto each flank of the memory OT-I chimera. P14 chimeras, that is mice housing P14 memory CD8 T cells, were generated by injecting naïve lymphocytic choriomeningitis virus (LCMV)-specific (P14) CD8+ T cells (5×10⁵) to B6 mice and 1 day later infecting i.p. with 2×10⁵ PFU LCMV-Armstrong.

Thy1.1 depletion. For Thy1.1 depletion experiments, a dose in a range of 0.625 μg to 3 μg of Thy1.1 (HIS51) antibody was injected intradermally with a Monoject 29G X½″ Insulin Safety Syringe or a Hamilton Syringe into one flank. An equal volume of IgG2a Isotype was injected into the opposite flank.

Antibody-based depletion of TRM. For the antibody-based depletion experiments, a dose in a range of 0.625 μg to 3 μg of antibody (see Table 2) was injected intradermally with a Monoject 29G X½″ Insulin Safety Syringe or a Hamilton Syringe into one flank. An equal volume of isotype control antibody (see Table 2) was injected into the opposite flank.

Immunofluorescence of the epidermis. Whole mounts of the epidermis were created as follows. Epidermal sheets were prepared from each flank of the treated mouse by affixing the epidermis side to slides with double sided adhesive (3M, Maplewood, Minn.). Slides were incubated with 2.5 mg/mL Dispase II in PBS for 45 minutes at 37° C. Epidermal mounts were fixed in chilled acetone for 5 minutes. The mounts were subsequently stained and images captured of TRM cells in epidermis were counted by ImageJ software.

### Results and Discussion

To investigate core and tissue specific CD8⁺ TRM cell surface markers, gene expression among diverse TRM populations was evaluated.

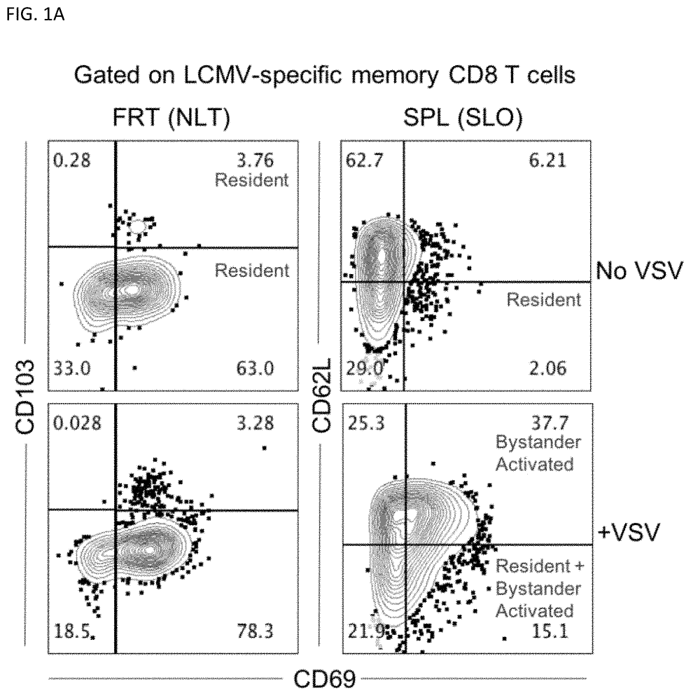

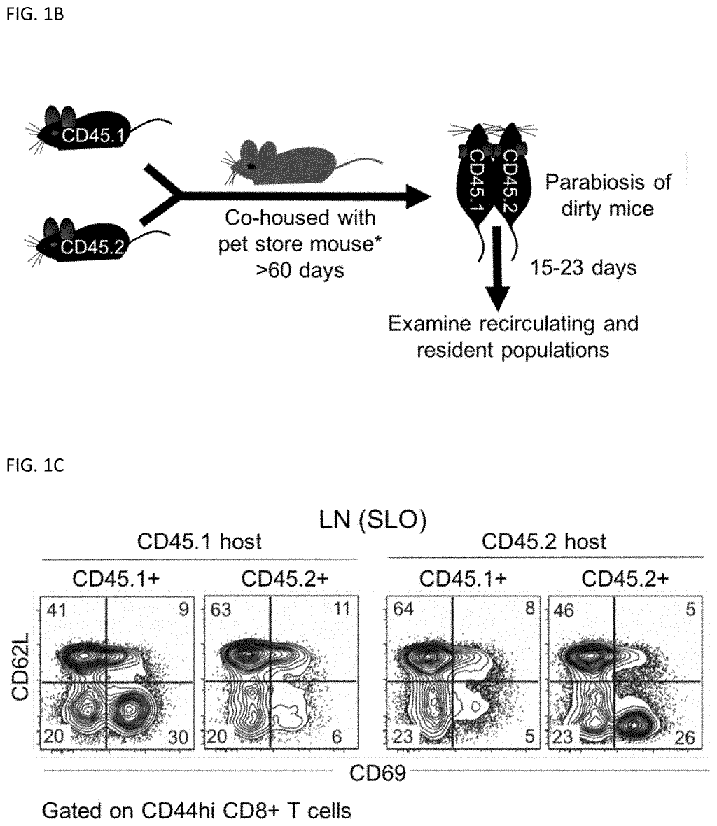

TRM are often defined by the cell surface expression of CD69, CD103, and/or CD49a, along with markers of antigen experience (for example, high expression of CD44 and/or low expression of CCR7) in both mouse and human studies (Topham et al. Front Immunol 9, 515 (2018)). CD69 has been reported as being expressed by the majority of CD4+ and CD8+ TRM cells in multiple sites (Kumar et al. Cell Rep 20, 2921-2934 (2017)). But, as shown in FIG. 1, CD69 and CD103 are not rigorously associated with tissue residence of TRM.

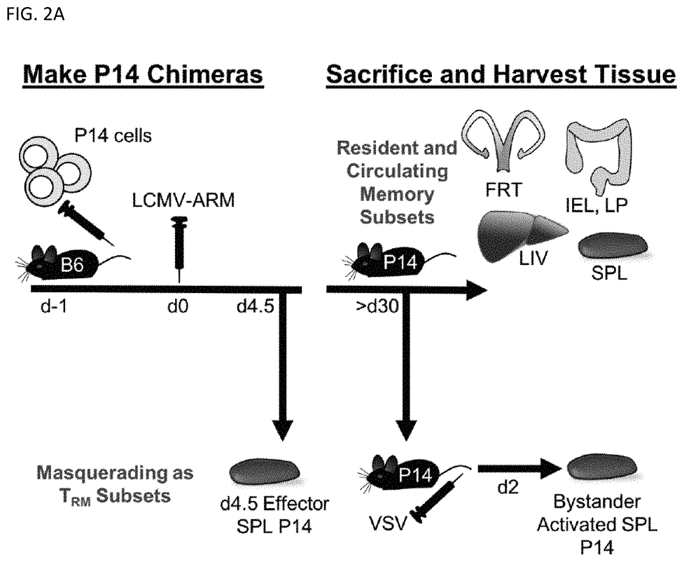

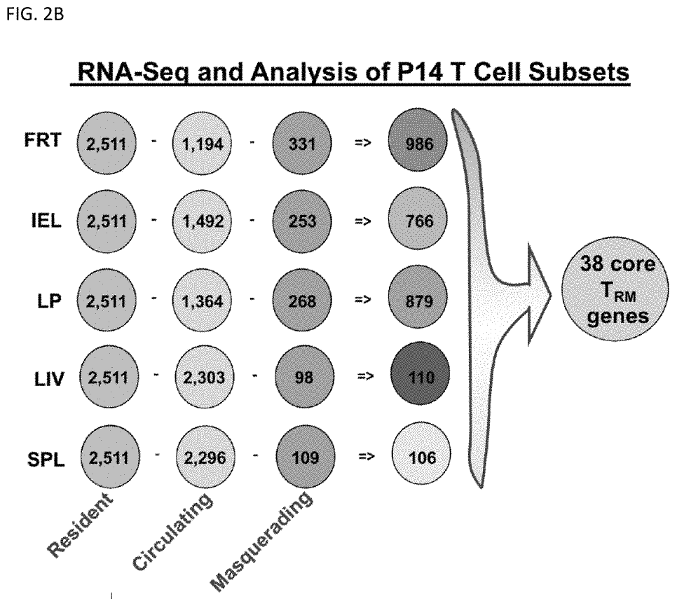

By comparing the gene expression of diverse TRM populations with recirculating T cell subsets as well as with T cell populations that transiently express the imperfect TRM biomarker CD69 in response to recent TCR stimulation or inflammation, CD8⁺ TRM-specific genes were identified. Results are shown in FIG. 2. See also Table 1.

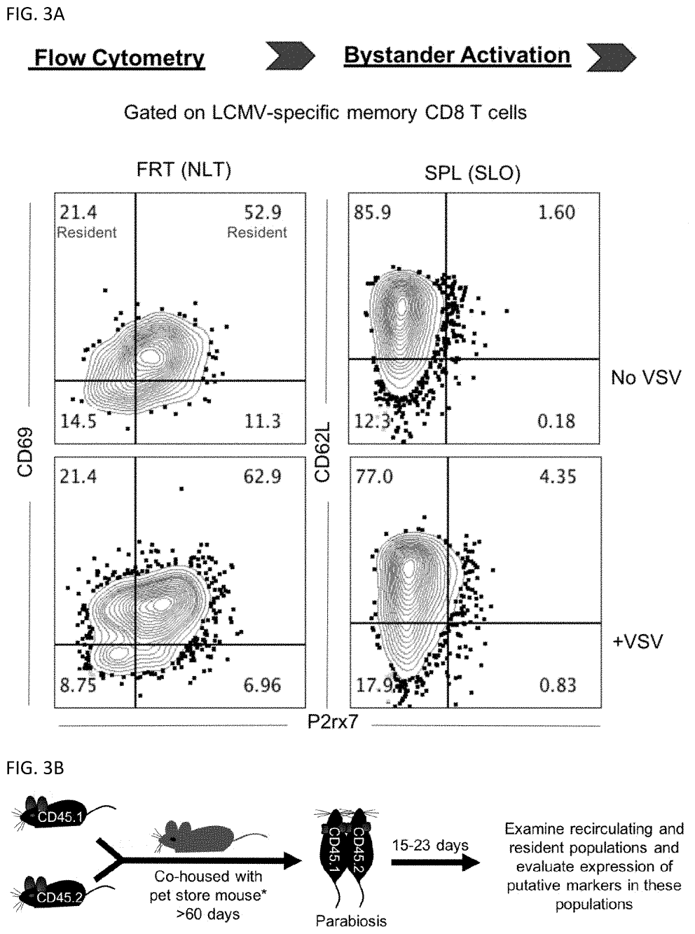

As shown in FIG. 3, the TRM biomarkers identified were validated via flow cytometry, parabiosis studies, and/or through the use of ‘dirty’ mice that contain CD69⁺ T cells within the equilibrating T cell population. Dirty mice have an immune system that is believed to more closely mimic the human immune system with respect to TRMs than typical laboratory mice (Beura et al. Nature 532, 512-516 (2016)).

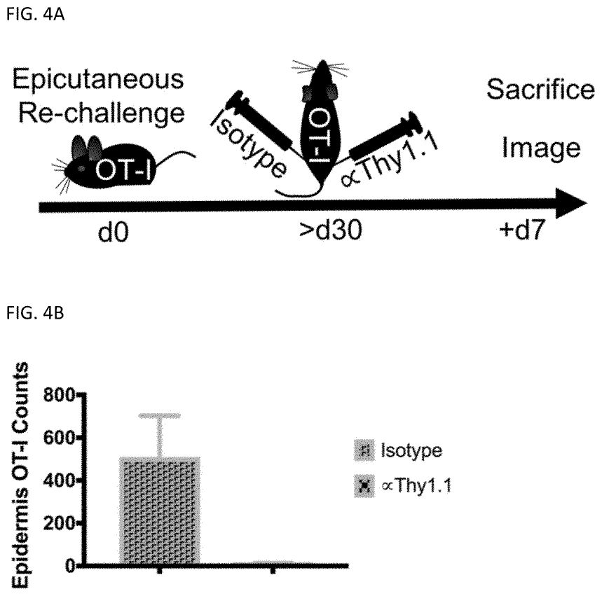

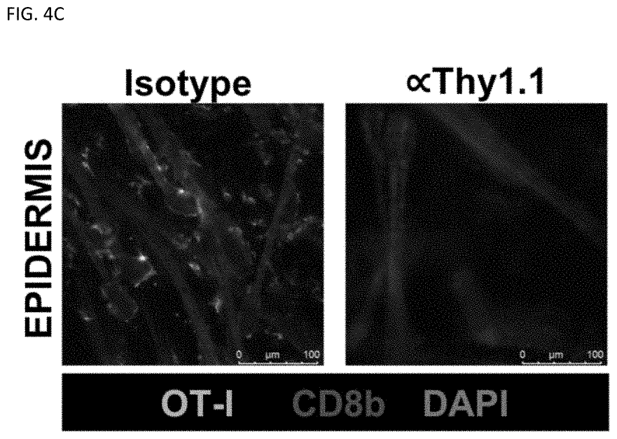

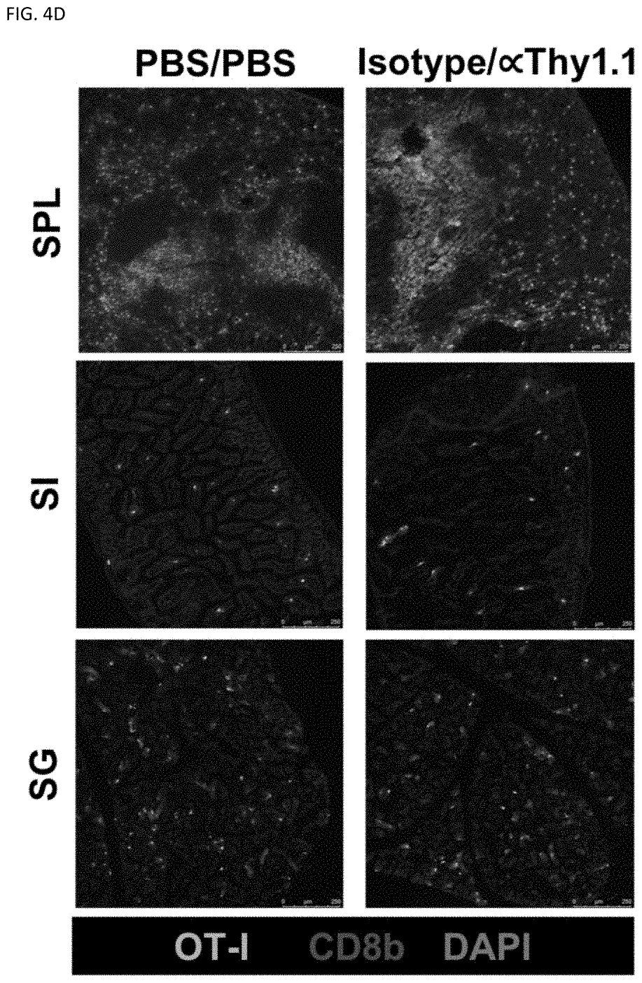

FIG. 4 shows a proof-of-concept study that shows that a marker specific to TRM in a mouse model can serve as a target for local depletion of epidermal TRM cells. Local depletion of Thy1.1⁺ TRM cells in the epidermis with anti-Thy1.1 antibody did not result in systemic effects. Systemic effects include, for example, depletion of TRM at alternative locations such as untreated skin or visceral organs (for example, the small intestine).

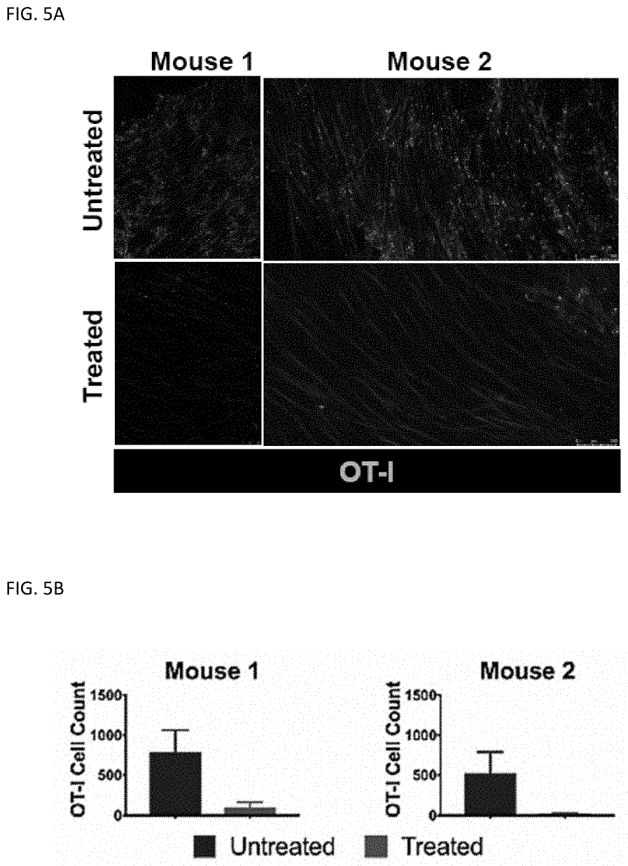

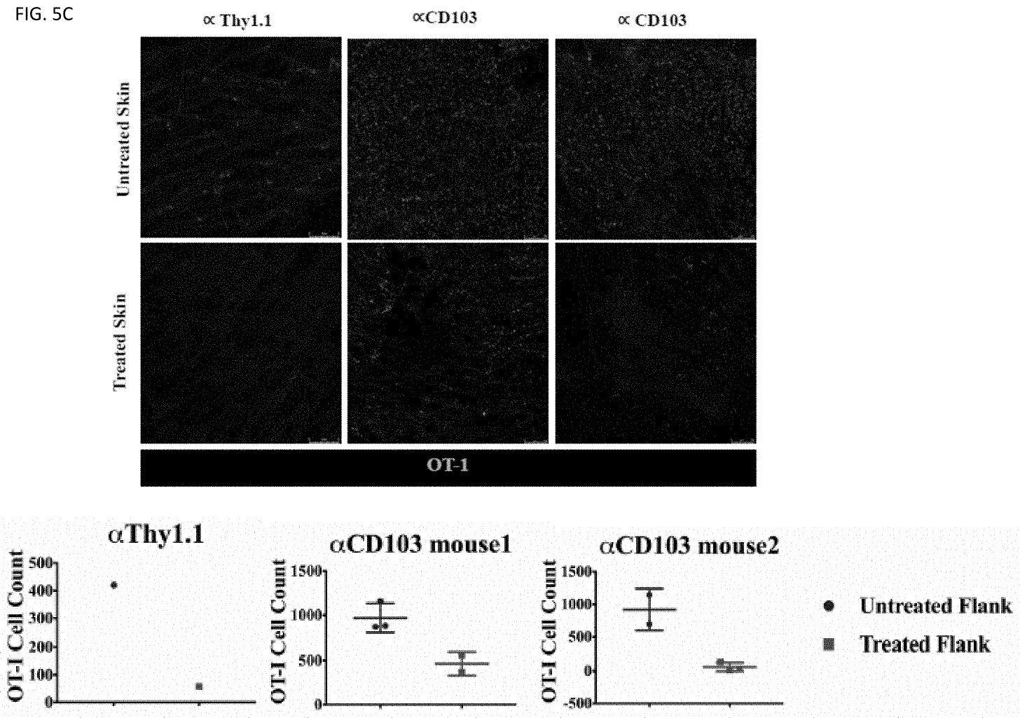

Local depletion of Thy1.1⁺ TRM cells in the epidermis with anti-Thy1.1 antibody treatment persisted 21 days after local antibody delivery (FIG. 5C).

In addition, when administered as described in the Materials and Methods but injecting Thy1.1 (HIS51) antibody intraperitoneally, systemic depletion of Thy1.1+ OT-I cells was observed with as little as 10 μg anti-Thy1.1 antibody.

### Example 2

This example describes proof of principle studies in mouse skin that demonstrate that antibodies to TRM-specific markers may locally deplete TRM. Materials and methods were as described in Example 1.

### Results and Discussion

Adult C57B1/6 mice were injected with CD45.1⁺ OTI CD8 T cells which recognize ova. The next day mice were infected with VSV-ova, which causes the induction of resident memory T cells (TRM) in many organs, including the skin.

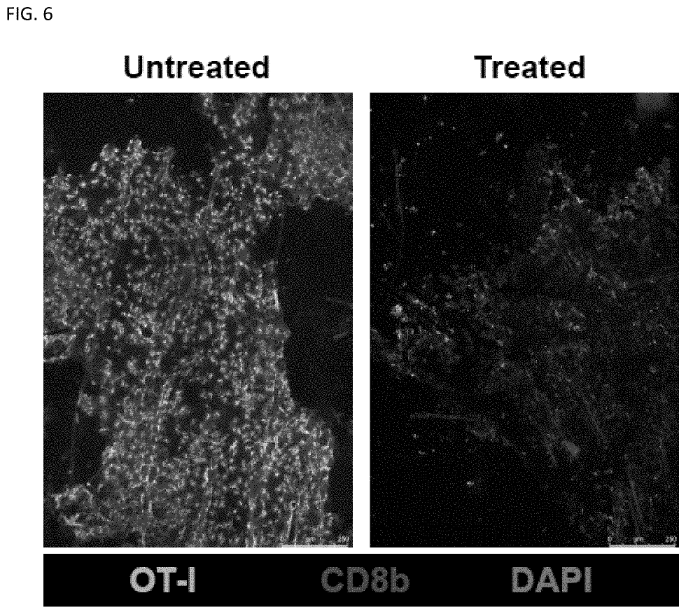

CD103 and CD49 have previously been demonstrated to be cell surface markers found on TRM in the skin. At 35 days after infection, the skin dermis of mice was injected with CD103 antibodies (FIG. 5) or CD49a antibodies (FIG. 6). 7 days after antibody injection, fluorescence microscopy was used to analyze mouse skin for the presence of OTI TRM (using a cell surface marker that is only on the injected OTI T cells). Results are shown in FIG. 5, FIG. 6, and Table 2. Local injection into the skin of an antibody targeting CD103 or an antibody targeting CD49a depletes TRM.

Using the antibody targeting CD103, depletion was apparent 6 days after antibody was intradermally injected and appeared to be durable; depletion was still observed 21 days after local antibody delivery (FIG. 5C).

TABLE 1

| CD8+ TRM Subsets | Circulating Memory Subsets | Masquerading Subsets |
|---|---|---|
| (CD69⁺, CD62L⁻, CD44⁺ memory P14s) | (CIR, CD69⁻, CD62L⁺, CD44⁺ memory P14s) | (MASQ, CD69⁺ non-TRM) |
| Non-Lymphoid Tissue (NLT) | Effector Memory (TEM) | LCMV D4 Effectors (LCMVD4) |
| Female Reproductive Tract (FRT) | Central Memory (TCM) | Bystanders (BYS) |
| Small Intestine Intraepithelial lymphocytes (IEL) | | |
| Small Intestine lamina propria lymphocytes (LP) | | |
| Secondary Lymphoid Organ (SLO) | | |
| Spleen (SP) | | |

TABLE 2

| | | Results (appears | | | | |
|---|---|---|---|---|---|---|
| Marker | TESTED | to deplete?) | Clone | Isotype | Company | Catalog No. |
| CD103 | Yes | Yes | 2E7 | Armenian Hamster IgG | BD Biosciences | 121406 |
| CD103 | Yes | No | M290 | LOU/M IgG2a | BD Biosciences | |
| CD69 | Yes | No | H1.2F3 | Armenian Hamster IgG | BD Biosciences | 104502 |
| CD49a | Yes | No | HMα1 | Armenian Hamster IgG | BD Biosciences | 142602 |
| CD49a | Yes | Yes | Ha31/8 | Armenian Hamster lgG2, λ1 | BD Biosciences | 142602 |

### Example 3

This Example describes validation of P2rx7 as a TRM marker.

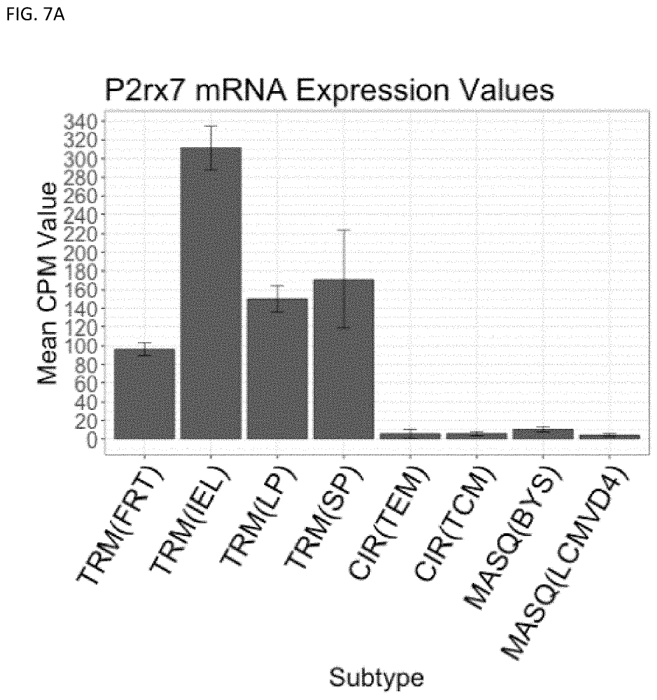

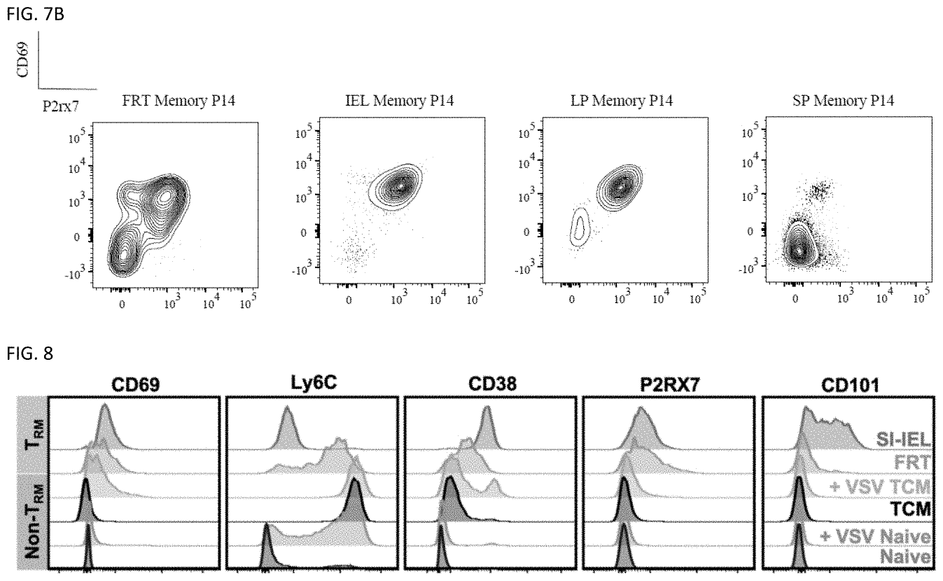

P2rx7 was one of the tissue-specific resident gene signatures identified in Example 1. mRNA expression values are shown in FIG. 7A. Protein expression of P2rx7 on memory P14 CD8 T cells after LCMV infection (as described in Example 4) was measured by flow cytometry; results are shown in FIG. 7B.

### Example 4

B6 mice were infected with LCMV-Armstrong 24 hours post-transfer of LCMV-specific cells. At >30 days after infection, mice were or were not infected with VSV. Two days later mice were sacrificed, and P14 cells were analyzed for TRM marker expression by fluorescence flow cytometry. Results are shown in FIG. 8.

These results indicate there are TRM markers, such as CD101, that are not expressed on bystander activated (VSV-induced) non-TRM CD8⁺ T cells.

### Example 5

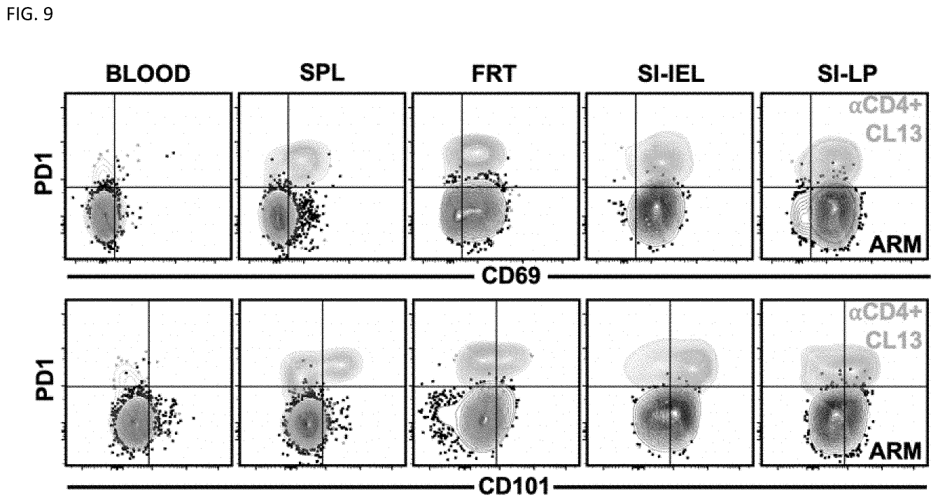

Expression of TRM markers in the context of chronic infection was examined. Results are shown in FIG. 9. Fluorescence flow cytometry plots of gp33 tetramer+ CD8+ T cells from mice at day 42 after infection with LCMV-Armstrong (Arm; antigen cleared) or with LCMV-CL13 (CL13; antigen not cleared) were analyzed.

The results indicate there are tissue specific CD8⁺ TRM markers, such as CD101, whose expression is induced on gp33 tetramer⁺ CD8⁺ T cells responding to chronic LCMV.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method comprising administering a composition to a subject suffering from a disease or condition, wherein the composition comprises a compound that binds to a resident memory T cell (TRM) marker.

2. The method of claim 1, wherein the TRM marker comprises CD103, CD49a, CCR8, CD38, CD39, NKG2D, CD69, CD101, PD-1, P2rx7, IL-23R, CD4, or CD8, or a combination thereof.

3. The method of claim 1, wherein the TRM marker is tissue specific.

4. The method of claim 1, wherein the compound that binds to a TRM marker comprises an antibody.

5. The method of claim 4, wherein the antibody comprises an antibody that fixes complement, an antibody that induces antibody dependent cell cytotoxicity (ADCC), or an antibody that induces phagocytosis of a resident memory T cell (TRM), or a combination thereof.

6. The method claim 1, wherein the disease or condition comprises an inflammatory immune response, an autoimmune disease, or an allergic disease, or a combination thereof.

7. The method claim 1, wherein the method comprises local delivery of the composition.

8. The method of claim 7, wherein the method comprises depleting the numbers of resident memory T cells (TRM) in the area where the composition was delivered.

9. The method of claim 8, wherein systemic TRM are decreased by less than 20%.

10. The method of claim 7, wherein the composition is formulated for oral, rectal, colonic, vaginal, topical, nasal, ophthalmic, and/or parenteral administration.

11. The method of claim 1, wherein the composition comprises a pharmaceutically acceptable carrier or an additional active agent or both.

12. A composition comprising a compound that binds to a resident memory T cell (TRM) marker, wherein the composition is formulated for oral, rectal, colonic, vaginal, topical, nasal, ophthalmic, and/or parenteral administration.

13. The composition of claim 12, wherein the composition comprises a pharmaceutically acceptable carrier or an additional active agent or both.

14. The composition of claim 12, wherein the TRM marker comprises CD103, CD49a, CCR8, CD38, CD39, NKG2D, CD69, CD101, PD-1, P2rx7, IL-23R, CD4, or CD8, or a combination thereof.

15. The composition of claim 12, wherein the compound that binds to a TRM marker comprises an antibody.

16. The composition of claim 15, wherein the antibody comprises an antibody that fixes complement, an antibody that induces antibody dependent cell cytotoxicity (ADCC), and/or an antibody that induces phagocytosis of a resident memory T cell (TRM).

17. A composition comprising a compound that binds to a resident memory T cell (TRM) marker for use in the treatment of a disease or condition comprising an inflammatory immune response.

18. The composition of claim 17, wherein the composition comprises a pharmaceutically acceptable carrier or an additional active agent or both.

19. The composition of claim 17, wherein the TRM marker comprises CD103, CD49a, CCR8, CD38, CD39, NKG2D, CD69, CD101, PD-1, P2rx7, IL-23R, CD4, or CD8, or a combination thereof.

20. The composition of claim 17, wherein the compound that binds to a TRM marker comprises an antibody.
